(19) United States
(12) Patent Application Publication
(10) Pub. No.: US 2013/0123540 A1
(43) Pub. Date: May 16, 2013

(54) METHOD FOR PRODUCING 2,5-DIAMINOTOLUENE

(71) Applicants: Otto Goettel, Marly (CH); Johann Aeby, Marly (CH)

(72) Inventors: Otto Goettel, Marly (CH); Johann Aeby, Marly (CH)

(73) Assignee: ALFA PARF GROUP S.P.A., Bologna (IT)

(21) Appl. No.: 13/811,740

(22) PCT Filed: Jul. 19, 2011
(86) PCT No.: PCT/EP11/62383

§ 371 Date: Jan. 23, 2013

(30) Foreign Application Priority Data
Jul. 23, 2010 (IT) MI2010A-001361

(51) Int. Cl. C07C 211/50; C07C 209/36

(52) U.S. Cl. CPC C07C 211/50; C07C 209/36
USPC 564/423; 564/420; 564/305

(57) ABSTRACT

The invention relates to a method for producing 2,5-diaminotoluene by catalytic hydrogenation in an aprotic-non-polar solvent or in a mixture made of two or more aprotic-nonpolar solvents with hydrogen in the presence of a catalyst or a mixture of two or more catalysts. The invention further relates to crystalline 2,5-diaminotoluene having characteristic crystal modification. As compared to conventional forms of administration common in industry, this is characterised in that it is largely insensitive to air oxidation.

The present invention concerns a method for the production of 2,5-diaminotoluene by catalytic hydrogenation in an aprotic-nonpolar solvent or in a mixture of two or more aprotic-nonpolar solvents with hydrogen in presence of a catalyst or a mixture of two or more catalysts. The present invention moreover concerns crystalline 2,5-diaminotoluene with a characteristic crystal modification. This is distinguished from traditional industrially used presentations by its substantial insensitivity to oxidation by air.

2,5-diaminotoluene of formula (I)

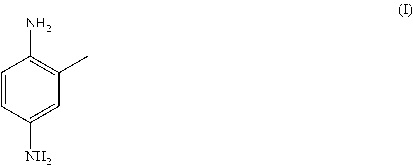

is used primarily in oxidative hair dyes in the cosmetic industry.

Commercially available 2,5-diaminotoluene, however, is a very oxidation-sensitive raw material. The high oxidation sensitivity of commercially available 2,5-diaminotoluene places considerable demands on its handling and storage and thus substantially limits the possibilities of using the 2,5-diaminotoluene in the form of the free base. Consequently, various experiments have been undertaken to stabilize 2,5-diaminotoluene with the goal of improving its handling and storage ability.

The most widespread stabilized form of 2,5-diaminotoluene at present is the 1:1 adduct product with sulfuric acid according to formula (II):

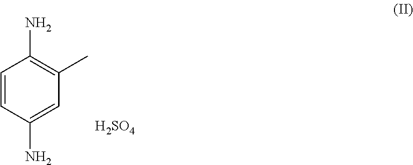

In the adduct with sulfuric acid, the amino groups of 2,5-diaminotoluene are protonated, so that the 2,5-diaminotoluene is sufficiently protected against oxidation by air and the storage capability is increased. The adduct does not change significantly, even over a long period of time, as long as it is kept cool and dry.

The easier handling and improved storage capability of the adduct of 2,5-diaminotoluene with sulfuric acid accordingly bring substantial advantages in practical application as compared to the use of 2,5-diaminotoluene in the free base form.

But if one uses the adduct of 2,5-diaminotoluene with sulfuric acid along with other components for the formulation of a hair dye, it often turns out that the salt burden attributable to the sulfuric acid bound in the adduct is a substantial disadvantage, especially when the hair dye formulations contain still other salts, such as those of color developer or coupler components. In especially unfavorable cases, such formulations can become unstable and separate out during storage, especially at elevated temperatures, becoming unusable. This behavior occurs especially in formulations with medium brown to black tints, which require larger dye proportions than other formulations.

In such unfavorable cases, manufacturers of hair dyes often resort to the use of 2,5-diaminotoluene in the form of its free base (I). The free base is commercially available either as a solidified melt in metal containers or as a stabilized aqueous solution with a content of around 25 to 50%. But in both instances the handling of such commercially available free 2,5-diaminotoluene imposes substantial technical demands.

Thus, for example, in those cases when the amorphous base needs to be used, the aforementioned metal containers are first heated above the melting point of 64° C. of the free base for its removal. But in the hot state the free base is especially sensitive to air, so that the removal needs to be done basically under inert conditions. Furthermore, there is the danger that when the heated free base makes contact with parts of the production layouts whose temperature lie below the melting point of the free base, very rapid solidification of the free base can occur, which in turn in unfavorable cases can lead to the clogging of the lines of the production layouts, for example.

If a manufacturer does not have a suitable installation available, especially for the removal of the free base melt, the marketplace offers him the alternative of aqueous solutions of 2,5-diaminotoluene. But when such aqueous solutions are used, runoff from these aqueous solutions of 2,5-diaminotoluene can form on the lids and the inner walls of the vessels used for the handling of the solutions, which become increasingly dark upon contact with traces of oxygen in the air of the gas space and thus again can contaminate the aqueous solutions of the free base. In this way, the aqueous solutions become increasingly darker, which generally has a negative effect on the color of the dye formulations.

Thus, for example, Japanese patent JP20000007622 describes the very high oxidation sensitivity of the solutions and points out the accelerated decomposition reactions once the solutions begin to oxidize. This can be determined visually by dark coloration of the solutions; as oxidation proceeds, the formation of tarry sediments can also be observed. The described oxidation processes can be sufficiently suppressed only at oxygen content below 10 ppm. Furthermore, the solutions must be stored in a cool and light-protected place.

Therefore, at present there is a need for the preparation of free 2,5-diaminotoluene in a form that is distinguished by easier handling and storage capability than traditional presentations.

Methods for the preparation of 2,5-diaminotoluene and its adducts are well known. As the educts, one can use, for example, azo dyes that form 2,5-diaminotoluene by cleavage of the azo group as well as a cleavage amine which then needs to be separated in the following purification steps. Alternatively to the cleavage of azo groups, one can also transform 2-amino-5-nitrotoluene reductively into 2,5-diaminotoluene.

2,5-diaminotoluene was first described by R. Nietzki (R. Nietzki, Chemische Berichte 10, 662 (1877)). Its preparation was accomplished by cleavage of o-amido-azotoluene by means of tin/hydrochloric acid to o-toluidine and 2,5-diaminotoluene. The product was conditioned by neutralizing the reaction mixture, extracting the two resulting bases with ether, fractionated distillation and recrystallization of the solidified 2,5-diaminotoluene. No details are mentioned, such as batch quantities, concentrations, yields or purities. As the product, Nietzki obtained “sheets grouped like a rosette”. Whether Nietzki was indeed able to isolate 2,5-diaminotoluene in crystalline form, especially in uniform crystalline form free of amorphous regions, cannot be found from his disclosure, due to the lack of an appropriate detection.

In more recent time, an Indian working group headed by D. Chane Gowda has dealt in numerous publications with possibilities of reducing azo compounds, which can be grouped into two main theme areas: first, methods that use metals like magnesium or zinc. Examples are cleavages of azo compounds by magnesium/ammonium formate (Abiraj, K.; Gowda, Shankare; Gowda, D. Channe, Journal of Chemical Research, Synopses, (5), 299-300; 2003) and cleavages of azo compounds by zinc/ammonium chloride in methanol (M. B. Sridhara; G. R. Srinivasa; D. Channe Gowda, Synthetic Communications 34 (8), 1441 (2004). Secondly, they have reported on possibilities of reducing azo compounds by hydrogen transfer reactions on nickel with ammonium formate (D. Channe Gowda et al., Indian Journal of Chemistry, Section B: Organic Chemistry Including Medicinal Chemistry, 42B(7), 1774-1776; 2003), as well as with hydrazine formate as the hydrogen source (H. S. Prasad; Shankare Gowda; D. Channe Gowda, Synthetic Communications, 34 (1), 1-10, 2004).

However, in the context of these methods, 2,5-diaminotoluene was not isolated as a free base, but as a benzoyl derivative.

Furthermore, a drawback in the synthesis methods known thus far from the prior art is that the conditioning of the obtained 2,5-diaminotoluene is costly and the described methods also for that reason are not suitable for production of the free base on an industrial scale. It is especially difficult to remove without a trace the nickel, as well as other metals and their salts, additional added salts, reagents such as free hydrazine or its adducts and the cleavage amines previously used for the diazotation, if the quality of the obtained 2,5-diaminotoluene is supposed to meet the criteria for use in cosmetics.

Removal of the cleavage amines can be bypassed by switching from the reduction of azo compounds to the reduction of aromatic nitro compounds, as was described in 2003 by Hari Sankar Kakati and Dibakar Chandra Deka (Indian Journal of Chemical Technology, 10(1), 60-62, 2003). The hydrogen transfer reaction with hydrazine hydrate on nickel catalyst at 150° C. produced 2,5-diaminotoluene in 42% yield. K. Bhaumik et al. also carried out the reduction on a nickel catalyst in the presence of ammonium chloride in water (K. Bhaumik and K. G. Akamanchi, Canadian Journal of Chemistry, 81(3), 197-198 (2003)).

However, in these cases as well, extractions of the base and the converting of the extracted base into the hydrochloride, followed by another liberating of the base, play a central role to ultimately realize yields of up to 86%. The quantities of solvents needed are considerable, so that these methods are unsuitable for production, due to ecological and economic reasons. Finally, in the mentioned methods, which primarily make use of Raney nickel as the catalyst, one must rule out traces of nickel, as well as hydrazine residues, from getting into the end product, if the quality of the obtained 2,5-diaminotoluene is supposed to meet the criteria for use in cosmetics.

Therefore, at present there exists a need for the providing of a method of production of the free 2,5-diaminotoluene in a form distinguished by easier handling and storage capability, especially as compared to traditional presentations, which is also suitable for industrial purposes from ecological and economic standpoints. In particular, there is a need at present for the providing of a method for production of free 2,5-diaminotoluene that is also applicable on an industrial scale.

The problem on which the present invention is based was to provide a method for the production of 2,5-diaminotoluene that does not have the drawbacks mentioned in this text, or only in diminished form. Therefore, the problem of the present invention was the providing of a method for the production of 2,5-diaminotoluene for use especially in cosmetics in a form distinguished by easier handling and storage capability, especially as compared to traditional presentations. A further problem of the present invention was to provide a method for production of 2,5-diaminotoluene that can be used especially in cosmetics and that can be used on an industrial scale, both from ecological and economic standpoints. From this standpoint, a particular problem of the present invention was to provide a method for production of 2,5-diaminotoluene that can be used especially in cosmetics and that especially avoids an entrainment of metal residues of various oxidation stages, of catalysts, of hydrazines and of salt burdens.

The problems are solved according to the invention by a method for the production of 2,5-diaminotoluene characterized in that 2-methyl-4-nitroaniline in an aprotic-nonpolar solvent or in a mixture of two or more aprotic-nonpolar solvents is hydrogenated with hydrogen in presence of a catalyst or a mixture of two or more catalysts to form a solution containing 2,5-diaminotoluene and an aprotic-nonpolar solvent or a mixture of two or more aprotic-nonpolar solvents.

A hydrogenation of the 2-methyl-4-nitroaniline in the sense of the present invention occurs for as long as the 2-methyl-4-nitroaniline takes up hydrogen in an aprotic-nonpolar solvent or in a mixture of two or more aprotic-nonpolar solvents.

One feature of the method of the invention is that the hydrogenation is carried out in an aprotic-nonpolar solvent or in a mixture of two or more aprotic-nonpolar solvents. The aprotic-nonpolar solvents or mixtures of two or more aprotic-nonpolar solvents described hereafter are generally distinguished in that they can be used both for the hydrogenation according to the invention and for the crystallization of the formed 2,5-diaminotoluene. The aprotic-nonpolar solvents or mixtures of two or more aprotic-nonpolar solvents described hereafter are furthermore generally distinguished in that they form azeotropes with water, which can be utilized after the hydrogenation for the drying of the solution containing 2,5-diaminotoluene and an aprotic-nonpolar solvent or a mixture of two or more aprotic-nonpolar solvents and therefore is of great advantage in the preparative chemistry. The skilled person, based on his general professional knowledge will distinguish the aprotic-nonpolar solvents described hereafter and the mixtures of two or more aprotic-nonpolar solvents described hereafter from aprotic-polar solvents and mixtures of two or more aprotic-polar solvents, as well as from protic solvents and mixtures of two or more protic solvents.

Suitable aprotic-nonpolar solvents according to the invention are known to the skilled person. One aprotic-nonpolar solvent in the sense of this text that is suitable according to the invention is distinguished in particular by its relative dielectric constant ∈ᵣ.

In the context of this text, the relative dielectric constant ∈ᵣ is defined, in a manner familiar to the skilled person, as the ratio of the dielectric constant ∈ᵣ and the electric field constant of a vacuum ∈₀:

∈ᵣ=∈ᵣ/∈₀

In the literature, the relative dielectric constant ∈ᵣ is also called alternatively the relative permittivity constant and the dielectric constant ∈ alternatively the permittivity constant.

According to the invention, suitable aprotic-nonpolar solvents are basically aprotic-nonpolar solvents whose relative dielectric constant ∈ᵣ^{20° C./100 kHz} measured at a temperature of 20° C. and a frequency of 100 kHz has a value of <8, such as 7 or <7 or <6.5 or ≦6.1, and >1, such as >2 or ≧3. The skilled person can find corresponding relative dielectric constants of solvents in relevant table collections, for example.

According to the invention, basically any aprotic-nonpolar solvent is suitable to carry out the hydrogenation if it allows one to accomplish the outcome of the invention. However, it is also provided by the invention to carry out the hydrogenation using a mixture of two or more aprotic-nonpolar solvents. Accordingly, any mixture of two or more aprotic-nonpolar solvents is basically suitable under the invention if it allows one to accomplish the outcome of the invention.

Thus, the hydrogenation according to the invention can be carried out, for example, if the aprotic-nonpolar solvent or the mixture of two or more aprotic-nonpolar solvents has a relative dielectric constant ∈ᵣ^{20° C./100 kHz} in the range of 2.3 to 6.1.

If the hydrogenation is carried out using only one aprotic-nonpolar solvent, then the invention calls for using as the aprotic-nonpolar solvent one with a relative dielectric constant ∈ᵣ^{20° C./100 kHz} in the range of 2.3 to 5.9, or in the range of 2.3 to 5.7, or in the range of 2.3 to 5.5, or in the range of 2.3 to 5.3, or in the range of 2.3 to 5.1, or in the range of 2.3 to 4.9, or in the range of 2.3 to 4.7, or in the range of 2.3 to 4.5, or in the range of 2.3 to 4.3, or in the range of 2.3 to 4.1, or in the range of 2.3 to 3.9, or in the range of 2.3 to 3.7, or in the range of 2.3 to 3.5, or in the range of 2.3 to 3.3, or in the range of 2.3 to 3.1, or in the range of 2.3 to 2.9, or in the range of 2.3 to 2.7, or in the range of 2.3 to 2.5, or in the range of 2.3 to 2.4 or preferably in the range of 2.38 to 2.39. Preferred according to the invention is, for example, an aprotic-nonpolar solvent with a relative dielectric constant ∈ᵣ^{20° C./100 kHz} of around 2.385.

Suitable aprotic-nonpolar solvents according to the invention are, for example, toluene, ethylbenzene, o-xylene, m-xylene, pseudocumene and tetralin. Preferred for carrying out the hydrogenation according to the invention is toluene.

If the hydrogenation is carried out using a mixture of two or more aprotic-nonpolar solvents, then the invention calls for using as the mixture of two or more aprotic-nonpolar solvents one with a relative dielectric constant ∈ᵣ^{20° C./100 kHz} in the range of 2.3 to 5.9, or in the range of 2.3 to 5.7, or in the range of 2.3 to 5.5, or in the range of 2.3 to 5.3, or in the range of 2.3 to 5.1, or in the range of 2.3 to 4.9, or in the range of 2.3 to 4.7, or in the range of 2.3 to 4.5, or in the range of 2.3 to 4.3, or in the range of 2.3 to 4.1, or in the range of 2.3 to 3.9, or in the range of 2.3 to 3.7, or in the range of 2.3 to 3.5, or in the range of 2.3 to 3.3, or in the range of 2.3 to 3.1, or in the range of 2.3 to 2.9, or preferably in the range of 2.30 to 2.70, such as in the range of 2.30 to 2.50, or in the range of 2.30 to 2.40, or in the range of 2.38 to 2.39. Suitable according to the invention is, for example, a mixture of two or more aprotic-nonpolar solvents with a relative dielectric constant of the mixture ∈ᵣ^{20° C./100 kHz} of around 2.385.

According to the invention, especially suitable for the production of mixtures of two or more aprotic-nonpolar solvents are aprotic-nonpolar solvents having a relative dielectric constant ∈ᵣ^{20° C./100 kHz} in the range of 2.3 to 6.1, such as those aprotic-nonpolar solvents that have already been indicated above. Suitable aprotic-nonpolar solvents according to the invention for the production of mixtures of two or more aprotic-nonpolar solvents are, for example, toluene, butylacetate, ethylacetate, ethylbenzene, o-xylene, m-xylene, pseudocumene and tetralin.

According to the invention, however, suitable mixtures of two or more aprotic-nonpolar solvents can also contain an aprotic-nonpolar solvent or two or more aprotic-nonpolar solvents each of which has a comparatively small relative dielectric constant ∈ᵣ^{20° C./100 kHz}, such as a relative dielectric constant ∈ᵣ^{20° C./100 kHz} of less than 2.3, provided that the mixture of two or more aprotic-nonpolar solvents has a relative dielectric constant ∈ᵣ^{20° C./100 kHz} in the range of 2.3 to 6.1, such as a relative dielectric constant ∈ᵣ^{20° C./100 kHz} in the range of 2.3 to 2.7.

Thus, according to the invention, a suitable mixture of two ore more aprotic-nonpolar solvents for carrying out the hydrogenation can contain only one aprotic-nonpolar solvent with a relative dielectric constant ∈ᵣ^{20° C./100 kHz} of less than 2.3, provided that the mixture of two or more aprotic-nonpolar solvents has a relative dielectric constant ∈ᵣ^{20° C./100 kHz} in the range of 2.3 to 6.1, such as a relative dielectric constant ∈ᵣ^{20° C./100 kHz} in the range of 2.3 to 2.7.

However, according to the invention, a suitable mixture of two ore more aprotic-nonpolar solvents for carrying out the hydrogenation can also contain two or three or more aprotic-nonpolar solvents each with a relative dielectric constant ∈ᵣ^{20° C./100 kHz} of less than 2.3, provided that the mixture of two or more aprotic-nonpolar solvents has a relative dielectric constant ∈ᵣ^{20° C./100 kHz} in the range of 2.3 to 6.1, such as a relative dielectric constant ∈ᵣ^{20° C./100 kHz} in the range of 2.3 to 2.7.

According to the invention, suitable for the production of a mixture of two or more aprotic-nonpolar solvents are aprotic-nonpolar solvents with a comparatively small relative dielectric constant ∈ᵣ^{20° C./100 kHz} such as cyclohexane, methylcyclohexane, p-xylene, mesitylene and p-cumene.

According to the invention, it is often preferable when the fraction of aprotic-nonpolar solvents with a relative dielectric constant ∈ᵣ^{20° C./100 kHz} of less than 2.3 in the mixture of two or more aprotic-nonpolar solvents is less than 5 wt. % or less than 4 wt. % or less than 3 wt. % or less than 2 wt. % or less than 1 wt. % or less than 0.5 wt. % or less than 0.1 wt. % or less than 0.05 wt. % or less than 0.01 wt. % or less than 0.001 wt. % or less than 0.0001 wt. %, each in terms of the total weight of the aprotic-nonpolar solvents. Often, however, it is preferable when the suitable mixture of two or more aprotic-nonpolar solvents according to the invention basically contains no aprotic-nonpolar solvent with a relative dielectric constant ∈ᵣ^{20° C./100 kHz} of less than 2.3.

Accordingly, suitable mixtures of two or more aprotic-nonpolar solvents according to the invention can also contain one aprotic-nonpolar solvent or two or more aprotic-nonpolar solvents each having a comparatively large relative dielectric constant ∈ᵣ^{20° C./100 kHz} such as a relative dielectric constant ∈ᵣ^{20° C./100 kHz} greater than 6.1, provided that the mixture of two or more aprotic-nonpolar solvents has a relative dielectric constant ∈ᵣ^{20° C./100 kHz} in the range of 2.3 to 6.1, such as a relative dielectric constant ∈ᵣ^{20° C./100 kHz} in the range of 2.3 to 2.7.

Thus, according to the invention, a suitable mixture of two ore more aprotic-nonpolar solvents for carrying out the hydrogenation can contain only one aprotic-nonpolar solvent with a relative dielectric constant ∈ᵣ^{20° C./100 kHz} of more ore than 6.1, provided that the mixture of two or more aprotic-nonpolar solvents has a relative dielectric constant ∈ᵣ^{20° C./100 kHz} in the range of 2.3 to 6.1, such as a relative dielectric constant ∈ᵣ^{20° C./100 kHz} in the range of 2.3 to 2.7.

However, according to the invention, a suitable mixture of two ore more aprotic-nonpolar solvents for carrying out the hydrogenation can also contain two or three or more aprotic-nonpolar solvents each with a relative dielectric constant ∈ᵣ^{20° C./100 kHz} of more than 6.1, provided that the mixture of two or more aprotic-nonpolar solvents has a relative dielectric constant ∈ᵣ^{20° C./100 kHz} in the range of 2.3 to 6.1, such as a relative dielectric constant ∈ᵣ^{20° C./100 kHz} in the range of 2.3 to 2.7.

According to the invention, it is often preferable when the fraction of aprotic-nonpolar solvents with a relative dielectric constant ∈ᵣ^{20° C./100 kHz} of more ore than 6.1 in the mixture of two or more aprotic-nonpolar solvents is less than 5 wt. % or less than 4 wt. % or less than 3 wt. % or less than 2 wt. % or less than 1 wt. % or less than 0.5 wt. % or less than 0.1 wt. % or less than 0.05 wt. % or less than 0.01 wt. % or less than 0.001 wt. % or less than 0.0001 wt. %, each in terms of the total weight of the aprotic-nonpolar solvents. Often, however, it is preferable when the suitable mixture of two or more aprotic-nonpolar solvents according to the invention basically contains no aprotic-nonpolar solvent with a relative dielectric constant ∈ᵣ^{20° C./100 kHz} of more than 6.1.

According to the invention, suitable mixtures of two or more aprotic-nonpolar solvents are, for example, mixtures comprising xylene/ethylbenzene/butylacetate, such as mixtures comprising technical-grade xylene/ethylbenzene/butylacetate. The respective fractions of xylene, ethylbenzene and butylacetate in a suitable mixture according to the invention of two or more aprotic-nonpolar solvents can vary in wide ranges.

Thus, for example, the fraction of xylene, especially technical-grade xylene, in a suitable mixture according to the invention of two or more aprotic-nonpolar solvents can lie in a range of 5 to 95 wt. % or in a range of 7.5 to 90 wt. % or in a range of 10 to 85 wt. % or in a range of 12.5 to 80 wt. % or in a range of 15 to 75 wt. % or in a range of 17.5 to 72.5 wt. % or in a range of 20 to 70 wt. %, each in terms of the total weight of the mixture of two or more aprotic-nonpolar solvents.

The fraction of ethylbenzene in a suitable mixture according to the invention of two or more aprotic-nonpolar solvents can lie in a range of 5 to 95 wt. % or in a range of 7.5 to 90 wt. % or in a range of 10 to 85 wt. % or in a range of 12.5 to 80 wt. % or in a range of 15 to 75 wt. % or in a range of 20 to 70 wt. % or in a range of 25 to 70 wt. %, each in terms of the total weight of the mixture of two or more aprotic-nonpolar solvents.

Finally, the fraction of butylacetate in a suitable mixture according to the invention of two or more aprotic-nonpolar solvents can lie in a range of 0.1 to 25 wt. % or in a range of 0.5 to 20 wt. % or in a range of 1 to 18 wt. % or in a range of 2 to 16 wt. % or in a range of 3 to 14 wt. % or in a range of 4 to 12 wt. % or in a range of 5 to 10 wt. %, each in terms of the total weight of the mixture of two or more aprotic-nonpolar solvents.

Especially suitable mixtures according to the invention are the mixture xylene/ethylbenzene/butylacetate in a ratio of 20 wt. % to 70 wt. % to 10 wt. % and the mixture xylene/ethylbenzene/butylacetate in a ratio of 70 wt. % to 25 wt. % to 5 wt. %, each in terms of the total weight of the mixture of two or more aprotic-nonpolar solvents.

According to the invention, suitable aprotic-nonpolar solvents for carrying out the hydrogenation can basically have any given boiling points, as long as the outcome of the invention can be achieved with them.

However, according to the invention it is preferred to select as the aprotic-nonpolar solvent one which has a boiling point of <200° C. or <175° C. or <150° C. or <120° C. or <100° C. or <90° C. or <85° C. under normal conditions.

Thus, according to the invention, suitable mixtures of two or more aprotic-nonpolar solvents for carrying out the hydrogenation can basically have any given boiling points, as long as the outcome of the invention can be achieved with them.

However, according to the invention it is preferred to select as the mixture of two or more aprotic-nonpolar solvents one which has a boiling point of <200° C. or <175° C. or <150° C. or <120° C. or <100° C. or <90° C. or <85° C. under normal conditions.

Finally, aprotic-nonpolar solvents and mixtures of two or more aprotic-nonpolar solvents are especially preferred to carry out the hydrogenation according to the invention that can easily be removed from the 2,5-diaminotoluene and recycled and thus also have ecological advantages. Often, according to the invention, it is most preferred to carry out the hydrogenation in an essentially pure aprotic-nonpolar solvent, and in this context essentially pure toluene should be mentioned explicitly.

The catalytic hydrogenation of 2-methyl-4-nitroaniline can essentially be done in any desired manner. However, preferable according to the invention is heterogeneous catalytic hydrogenation of 2-methyl-4-nitroaniline, especially since in the context of the conditioning of the obtained solution containing 2,5-diaminotoluene and an aprotic-nonpolar solvent or a mixture of two or more aprotic-nonpolar solvents the catalyst or the mixture of two or more catalysts can be separated easily and generally essentially free of residue from them in a way known to the skilled person and essentially recycled. According to the invention, the catalysts known to the skilled person that are customarily used in hydrogenation processes or also in transfer hydrogenations are generally suitable.

Accordingly, the invention provides that the catalyst is a solid catalyst or the mixture of two or more catalysts is a mixture containing at least one solid catalyst or the mixture of two or more catalysts is a mixture containing only solid catalysts.

According to the invention, suitable solid catalysts are basically those based on nickel, such as Raney nickel, those based on cobalt, such as Raney cobalt, and those based on precious metals, such as palladium or platinum. According to the invention, solid catalysts based on precious metals like palladium or platinum are often preferred.

The catalyst can be, for example, a filled catalyst and accordingly consist, for example, entirely of palladium or platinum or both. According to the invention, the powder catalysts, shaped body catalysts and monolithic catalysts known to the skilled person and suitable for hydrogenation can basically be used for the hydrogenation of 2-methyl-4-nitroaniline. However, the catalyst can also be a supported catalyst and accordingly contain the palladium or the platinum or both in supported form.

Supported catalysts are often preferred. In the context of the present invention, suitable support materials are, for example, charcoal, activated charcoal, aluminum oxide, silicon dioxide, titanium dioxide, cordierite, zeolite, calcium carbonate and barium sulfate or mixtures of two or more of these.

Thus, for example, the invention provides for using palladium supported on charcoal, on activated charcoal, on aluminum oxide, on silicon dioxide, on titanium dioxide, on cordierite, on zeolite, on calcium carbonate or on barium sulfate as the catalyst for hydrogenation of 2-methyl-4-nitroaniline. Supported palladium catalysts or supported platinum catalysts are often preferred.

According to the invention, often specially preferred as solid catalyst is a palladium/charcoal catalyst, i.e., a catalyst that contains palladium, especially metallic palladium, supported on charcoal, especially supported on activated charcoal. The content of palladium can vary in broad ranges. According to the invention, palladium/activated charcoal catalysts can be used for the hydrogenation of 2-methyl-4-nitroaniline with a content of palladium in the range of 0.01 to 30 wt. % or in the range of 0.05 to 25 wt. % or in the range of 0.1 to 20 wt. % or in the range of 0.5 to 18 wt. % or in the range of 1 to 16 wt. % or in the range of 3 to 14 wt. % or in the range of 4 to 12 wt. % or in the range of 5 to 10 wt. %, each in terms of the total weight of the palladium/activated charcoal catalyst.

For safety reasons, according to the invention it is preferable to use the catalysts water-moistened with a water content in the range of 30 to 70 wt. %, for example, in the range of 35 to 65 wt. % or in the range of 40 to 60 wt. % or in the range of 45 to 55 wt. % or in the range of 47 to 53 wt. % or in the range of 48 to 52 wt. % or in the range of 49 to 51 wt. % or in the range of 49.5 to 50.5 wt. % or in the range of 49.8 to 50.2 wt. % or in the range of 49.9 to 50.1 wt. %, each in terms of the total weight of the water-moistened catalyst, such as the water-moistened palladium/activated charcoal catalyst. In the context of special embodiments of the present invention, water-moistened palladium/activated charcoal catalysts are used with a water content of around 50 wt. % in terms of the total weight of the water-moistened palladium/activated charcoal catalyst.

The quantity of the catalyst used in the given case for hydrogenation of 2-methyl-4-nitroaniline according to the invention can be varied within broad limits.

For example, a suitable moist catalyst according to the invention, for example a moist palladium/active carbon catalyst, in a quantity in the range of 0.1 to 20 wt % or in the range of 0.5 to 16 wt % or in the range of 0.9 to 12 wt % or in the range of 1 to 10 wt %, especially in the range of 6 to 10 wt %, in each case based on the total weight of the 2-methylnitroaniline, may be used.

The hydrogenation according to the invention is frequently preceded by the preparation of a mixture containing 2-methyl-4-nitroaniline, an aprotic-nonpolar solvent, or a mixture of two or more aprotic-nonpolar solvents and a catalyst or a mixture of two or more catalysts. The production of this mixture can basically follow the method known to a person skilled in the art.

However, according to the invention it is preferred first to dissolve or suspend 2-methyl-4-nitroaniline in an aprotic-nonpolar solvent or in a mixture of two or more aprotic-nonpolar solvents, for example under agitation, and then add the catalyst or the mixture of two or more catalysts.

The hydrogenation of the 2-methyl-4-nitroaniline with hydrogen can also basically be done in any arbitrary fashion known to a person skilled in the art.

However, according to the invention it is preferred first to bring the mixture containing 2-methyl-4-nitroaniline, an aprotic-nonpolar solvent or a mixture of two or more aprotic-nonpolar solvents and a catalyst or a mixture of two or more catalysts to a temperature suitable for hydrogenation, to initiate the hydrogenation by applying a positive hydrogen pressure, and to keep the temperature in a suitable range during hydrogenation.

In this process, the hydrogenation of the 2-methyl-nitroaniline with hydrogen basically can be performed in a broad temperature range.

For example, it is possible to perform the hydrogenation of the 2-methyl-4-nitroaniline in a temperature range of 0° C. to 150° C., for example in a range of 10° C. to 140° C. or from 20° C. to 130° C. or from 30° C. to 120° C. or from 40° C. to 110° C. or from 50° C. to 100° C. or from 60° C. to 90° C., especially in the range from 60° C. to 85° C.

In this process it is possible and provided first to bring a mixture containing 2-methyl-4-nitroaniline, an aprotic-nonpolar solvent or a mixture of two or more aprotic-nonpolar solvents and a catalyst or a mixture of two or more catalysts, especially by cooling or heating, to a temperature of at least 0° C. or at least 10° C. or at least 20° C. or at least 30° C. or at least 40° C. or at least 50° C. or at least or at least 60° C. or at least or at least 70° C. or at least 80° C. or at least 90° C. or at least 100° C. or at least 110° C. or at least 120° C. or at least 130° C. or at least 140° C. or at most 149.9° C. and then to start the hydrogenation of the 2-methyl-4-nitroaniline with hydrogen.

The initiation of the hydrogenation of the 2-methyl-4-nitroaniline can be performed by applying a positive pressure of gaseous hydrogen to the mixture containing 2-methyl-4-nitroaniline, an aprotic-nonpolar solvent or a mixture of two or more aprotic-nonpolar solvents and a catalyst or a mixture of two or more catalysts.

For example it may be provided to initiate the hydrogenation of the 2-methyl-4-nitroaniline according to the invention by applying a positive hydrogen pressure in the range of 0 to 5 MPa, for example by applying a positive hydrogen pressure in the range of 0 to 4 MPa or in the range of 0 to 3 MPa or in the range of 0 to 2 MPa or in the range of 0 to 1 MPa.

According to the invention, a positive hydrogen pressure in the range of 0.01 to 0.9 MPa or in the range of 0.02 to 0.8 MPa or in the range of 0.03 to 0.7 MPa or in the range of 0.04 to 0.6 MPa, especially or in the range of 0.05 to 0.6 MPa or in the range of 0.1 to 0.6 MPa or in the range of 0.2 to 0.6 MPa or in the range of 0.3 to 0.5 MPa is frequently and preferably used for initiating the hydrogenation of the 2-methyl-4-nitroaniline.

After the hydrogenation is started, an increase in the temperature of the mixture containing 2-methyl-4-nitroaniline, an aprotic-nonpolar solvent or a mixture of two or more aprotic-nonpolar solvents and a catalyst or a mixture of two or more catalysts regularly begins.

However, according to the invention it is possible and provided, after initiation of hydrogenation, to limit the temperature increase by cooling, especially by external cooling, in a manner known to the person skilled in the art, for example to a temperature of no more than 150° C. or of no more than 140° C. or of no more than 130° C. or of no more than 120° C. or of no more than 110° C. or of no more than 100° C. or of no more than 90° C. or of no more than 85° C. or of no more than 80° C. or of no more than 70° C. or of no more than 60° C. or of no more than 50° C. or of no more than 40° C. or of no more than 30° C. or of no more than 20° C. or of no more than 10° C. or of no less than 0.1° C.

Following the initiation of the hydrogenation, the further hydrogenation of the 2-methyl-4-nitroaniline with hydrogen basically can take place within a broad range of positive hydrogen pressures.

For example, it is provided that the 2-methyl-4-nitroaniline is hydrogenated at a positive hydrogen pressure in the range of 0 to 5 MPa, for example at a positive hydrogen pressure in the range of 0 to 4 MPa or 0 to 3 MPa or 0 to 2 MPa or 0 to 1 MPa.

Frequently preferred for hydrogenation of the 2-methyl-4-nitroaniline according to the invention is a positive hydrogen pressure in the range of 0.01 to 0.9 MPa or in the range of 0.02 to 0.8 MPa or in the range of 0.03 to 0.7 MPa or in the range of 0.04 to 0.6 MPa, especially in the range of 0.05 to 0.6 MPa or in the range of 0.1 to 0.6 MPa or in the range of 0.2 to 0.6 MPa or in the range of 0.3 to 0.5 MPa.

After the hydrogenation is initiated, the positive hydrogen pressure can essentially be kept constant during the remainder of the hydrogenation by measures known to the person skilled in the art. However, after initiation of the hydrogenation, at first a decrease in the positive hydrogen pressure may occur because of the onset of hydrogen consumption. Nevertheless it is likewise provided that after initiation of the hydrogenation, the positive hydrogen pressure may be systematically varied within one of the above-mentioned ranges during the further hydrogenation.

After the hydrogenation of the 2-methyl-4-nitroaniline is completed, the mixture obtained by the hydrogenation of the 2-methyl-4-nitroaniline according to the invention, containing 2,5-diaminotoluene, an aprotic-nonpolar solvent or a mixture of two or more aprotic-nonpolar solvents and a catalyst or a mixture of two or more catalysts, may take place. Workup according to the invention may comprise, for example, removal of the catalyst or the mixture of two or more catalysts from the mixture formed by the hydrogenation of 2-methyl-4-nitroaniline according to the invention, containing 2,5-diaminotoluene and an aprotic-nonpolar solvent or a mixture of two or more aprotic-nonpolar solvents. However, workup according to the invention may also comprise the removal of water from the mixture formed by the hydrogenation of 2-methyl-4-nitroaniline, containing 2,5-diaminotoluene and an aprotic-nonpolar solvent or a mixture of two or more aprotic-nonpolar solvents. If a workup according to the invention comprises both the removal of the catalyst or the mixture of two or more catalysts and the removal of water, the removal of the catalyst or the mixture of two or more catalysts may take place before the removal of water. However, it is also provided according to the invention that the removal of water may take place before the removal of the catalyst or the mixture of two or more catalysts.

Thus it is possible and provided according to the invention to remove the catalyst or the mixture of two or more catalysts from the solution containing 2,5-diaminotoluene and an aprotic-nonpolar solvent or a mixture of two or more aprotic-nonpolar solvents formed by the hydrogenation of 2-methyl-4-nitroaniline according to the invention.

The removal of the catalyst or the mixture of two or more catalysts from the solution containing 2,5-diaminotoluene and an aprotic-nonpolar solvent or a mixture of two or more aprotic-nonpolar solvents formed by the hydrogenation of 2-methyl-4-nitroaniline according to the invention basically can be performed in a manner known to the person skilled in the art.

However, it is frequently preferred that the catalyst or the mixture of two or more catalysts be separated from the solution containing 2,5-diaminotoluene and an aprotic-nonpolar solvent or a mixture of two or more aprotic-nonpolar solvents formed by the hydrogenation of the 2-methyl-4-nitroaniline according to the invention by using a filter, for example a pressure filter.

In this process it is preferred according to the invention if the filter, for example a pressure filter, is preheated to a temperature in the range of 50° C. to 150° C. or in the range of 52° C. to 140° C. or in the range of 53° C. to 130° C. or in the range of 54° C. to 120° C. or in the range of 55° C. to 110° C. or in the range of 56° C. to 100° C. or in the range of 57° C. to 95° C. or in the range of 58° C. to 90° C. or in the range of 59° C. to 85° C. or in the range of 60° C. to 80° C.

Furthermore it is possible and intended according to the invention that the solution containing 2,5-diaminotoluene and an aprotic-nonpolar solvent or a mixture of two or more aprotic-nonpolar solvents formed by the hydrogenation of 2-methyl-4-nitroaniline according to the invention be conducted into a preheated vessel before or after the filtration, for example before or after the pressure filtration.

In this process it is preferred according to the invention if the vessel is preheated to a temperature in the range of 50° C. to 150° C. or in the range of 52° C. to 140° C. or in the range of 53° C. to 130° C. or in the range of 54° C. to 120° C. or in the range of 55° C. to 110° C. or in the range of 56° C. to 100° C. or in the range of 57° C. to 95° C. or in the range of 58° C. to 90° C. or in the range of 59° C. to 85° C. or in the range of 60° C. to 80° C.

It is furthermore provided according to the invention that water be removed from the solution formed by the hydrogenation of 2-methyl-4-nitroaniline according to the invention, containing 2,5-diaminotoluene and an aprotic-nonpolar solvent or a mixture of two or more aprotic-nonpolar solvents, before or after filtration, for example before or after pressure filtration. This water may, for example, be water of reaction. Depending on the specific conditions selected for hydrogenation of the 2-methyl-4-nitroaniline, however, this may also relate to the water content of a moist catalyst used for hydrogenation of 2-methyl-4-nitroaniline.

The removal of water can basically be done in a manner known to a person skilled in the art. However, it is frequently preferred if the water is removed from the solution, formed by the hydrogenation of 2-methyl-4-nitroaniline according to the invention, containing 2,5-diaminotoluene and an aprotic-nonpolar solvent or a mixture of two or more aprotic-nonpolar solvents at a temperature in the range of 50° C. to 150° C. or in the range of 52° C. to 140° C. or in the range of 53° C. to 130° C. or in the range of 54° C. to 120° C. or in the range of 55° C. to 110° C. or in the range of 56° C. to 100° C. or in the range of 57° C. to 95° C. or in the range of 58° C. to 90° C. or in the range of 59° C. to 85° C. or in the range of 60° C. to 80° C. by applying a vacuum, for example a slight vacuum in the range of about 10 mbar to about 200 mbar or about 60 mbar to about 120 mbar. In this process, adequate removal of the water is often already achieved by azeotropic drying in view of the fact that the water and the aprotic-nonpolar solvent or the mixture of two or more aprotic-nonpolar solvents used for producing 2,5-diaminotoluene according to the invention regularly form azeotropes.

In addition, it is frequently preferred that the water be removed from the solution, formed by the hydrogenation of 2-methyl-4-nitroaniline according to the invention, containing 2,5-diaminotoluene and an aprotic-nonpolar solvent or a mixture of two or more aprotic-nonpolar solvents, using a water separator.

After removing the water, the vacuum can be broken, for example, by introducing nitrogen or another inert or essentially inert gas.

Furthermore it is provided according to the invention that 2,5-diaminotoluene be removed from the solution formed containing 2,5-diaminotoluene and an aprotic-nonpolar solvent or a mixture of two or more aprotic-nonpolar solvents.

The removal of the 2,5-diaminotoluene from the solution formed containing 2,5-diaminotoluene and an aprotic-nonpolar solvent or a mixture of two or more aprotic-nonpolar solvents is accomplished, for example, after the above-described removal of water following cooling of the solution formed containing 2,5-diaminotoluene and an aprotic-nonpolar solvent or a mixture of two or more aprotic-nonpolar solvents to a temperature in the range of less than 50° C., for example following cooling of the solution formed to a temperature in the range from 10° C. to 49° C. or from 20° C. to 48° C. or from 30° C. to 47° C. or from 40° C. to 46° C. or from 44.5° C. to 45.5° C.

Finally, according to the invention the precipitated 2,5-diaminotoluene is separated from the aprotic nonpolar solvent or from the mixture made of two or more aprotic nonpolar solvents. If desired, the 2,5-diaminotoluene separated from the aprotic nonpolar solvent or from the mixture made of two or more aprotic nonpolar solvents can be dried. The precipitation and drying of the 2,5-diaminotoluene can be carried out in each case using methods that are known to a person skilled in the art.

The present invention thus also relates to methods for producing 2,5-diaminotoluene, which are characterized in that at least one of the following steps is carried out after the hydrogenation:
the catalyst or the mixture made of two or more catalysts is separated from the solution that has been formed;
water is separated from the solution that has been formed;
the temperature of the solution that has been formed is maintained in a range in which 2,5-diaminotoluene is precipitated from the solution that has been formed;
the precipitated 2,5-diaminotoluene is separated from the solvent or from the mixture made of two or more solvents; and
the precipitated 2,5-diaminotoluene is dried.

Preferred embodiments of the method according to the invention for producing 2,5-diaminotoluene are characterized in that, after the hydrogenation
the catalyst or the mixture made of two or more catalysts and
water are separated from the solution that has been formed;
the temperature of the solution that has been formed is maintained in a range in which 2,5-diaminotoluene is precipitated from the solution that has been formed;
the precipitated 2,5-diaminotoluene is separated from the solvent or from the mixture made of two or more solvents and
is dried.

Frequently, it is further preferred when the precipitation takes place with crystallization of 2,5-diaminotoluene.

In accordance with the examples described within the scope of this specification, bringing about the crystallization of the 2,5-diaminotoluene does not pose any demands on a person skilled in the art that go beyond the common general knowledge in the art of this person. However, it is frequently advantageous to bring about the crystallization of the 2,5-diaminotoluene when the weight ratio between 2,5-diaminotoluene and the aprotic nonpolar solvent or the mixture made of two or more aprotic nonpolar solvents is in a range of 1:3 to 1:10 or in a range of 1:3,5 to 1:8 or in a range of 1:4 to 1:6. The crystalline 2,5-diaminotoluene produced by the method according to the invention has very high purity and is generally obtained in a yield of more than 85%.

The crystalline 2,5-diaminotoluene produced by the method according to the invention significantly differs with regard to the crystal habit thereof from the rosette-like grouped tables as described by R. Nietzki (R. Nietzki, Chemische Berichte (Chemical Reports) 10, 662, (1877)).

When using Co—K_{α1} radiation, crystalline 2,5-diaminotoluene produced according to the invention shows the strongest reflex in an x-ray powder diffractogram in a range of 2Θ=21.5° to 22.5°, the second-strongest reflex in a range of 2Θ=30.7° to 31.7°, and the third-strongest reflex in a range of 2Θ=24.1° to 25.1°. A corresponding peak list of an x-ray powder diffractogram of 2,5-diaminotoluene is shown in Table 1.

TABLE 1
Peak list of an x-ray powder diffractogram, obtained with Co—K_{α1}
radiation, of 2,5-diaminotoluene, produced by the method according
to the invention using toluene.
Peak list [range 1: 2Theta = 2.000 59.980 0.020 Imax = 33054]

| D | 2Theta | I (rel) | I (abs) | I (int) | FWHM |
|---|---|---|---|---|---|
| 9.375402 | 10.9495 | 5.46 | 1825 | 0.00 | 0.1000 |
| 6.659041 | 15.4393 | 25.21 | 8432 | 0.00 | 0.1000 |
| 4.698379 | 21.9501 | 100.00 | 33450 | 0.00 | 0.1000 |
| 4.200428 | 24.5906 | 37.59 | 12574 | 0.00 | 0.1000 |
| 3.840514 | 26.9366 | 21.22 | 7096 | 0.00 | 0.1000 |
| 3.562174 | 29.0859 | 9.74 | 3258 | 0.00 | 0.0800 |
| 3.323904 | 31.2222 | 64.39 | 21537 | 0.00 | 0.1000 |
| 3.130074 | 33.2098 | 14.07 | 4706 | 0.00 | 0.1000 |
| 2.972442 | 35.0264 | 27.50 | 9200 | 0.00 | 0.1000 |
| 2.839287 | 36.7262 | 3.58 | 1197 | 0.00 | 0.1000 |
| 2.605393 | 40.1585 | 7.02 | 2347 | 0.00 | 0.1200 |
| 2.512256 | 41.7153 | 4.78 | 1599 | 0.00 | 0.2000 |
| 2.428860 | 43.2182 | 6.92 | 2316 | 0.00 | 0.1000 |
| 2.350052 | 44.7445 | 4.30 | 1437 | 0.00 | 0.1200 |
| 2.278239 | 46.2353 | 1.63 | 545 | 0.00 | 0.1400 |
| 2.222108 | 47.4738 | 2.09 | 698 | 0.00 | 0.1200 |
| 2.158290 | 48.9681 | 2.28 | 762 | 0.00 | 0.1400 |
| 2.104975 | 50.2934 | 2.04 | 683 | 0.00 | 0.2200 |
| 2.004442 | 53.0067 | 8.36 | 2797 | 0.00 | 0.1600 |
| 1.918906 | 55.5683 | 4.97 | 1661 | 0.00 | 0.1600 |
| 1.886432 | 56.6102 | 2.73 | 912 | 0.00 | 0.1200 |
| 1.845479 | 57.9841 | 4.64 | 1552 | 0.00 | 0.1800 |

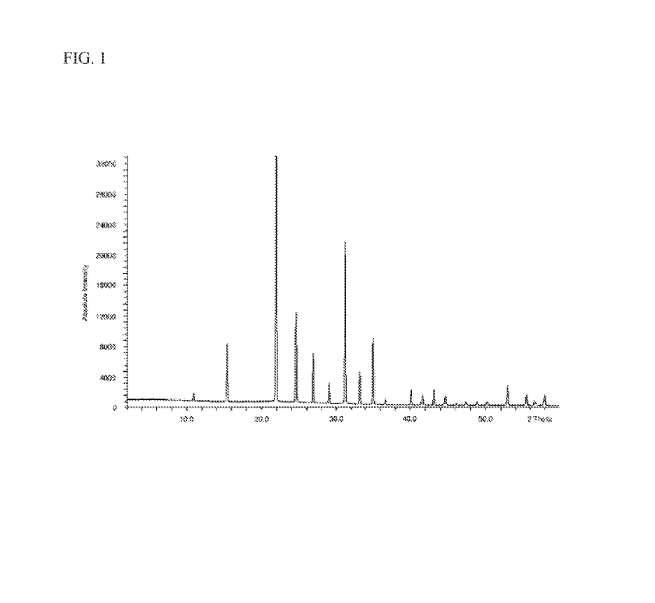

The corresponding x-ray powder diffractogram is shown in FIG. 1.

The crystalline 2,5-diaminotoluene produced according to the invention is characterized in particular in that it is substantially insensitive toward oxidation by air and therefore easy to handle. The crystalline 2,5-diaminotoluene produced according to the invention can thus be used excellently for producing cosmetics, in particular hair dye formulations. The crystalline 2,5-diaminotoluene produced according to the invention can additionally be used to produce plastic materials, in particular high-purity polyamides, and to produce liquid crystals.

The present invention will be described in more detail hereafter based on examples.

### EXAMPLE 1

Hydrogenation: 1521.5 g (10 mol) 2-amino-5-nitrotoluene (purity: >99.0%) is added to 15 liters of toluene in a nitrogen atmosphere and stirred for 30 minutes at room temperature. Then, 150 g palladium 5% on carbon (water content 50%, corresponds to 75 g dried product) is added. The batch is then heated to an internal temperature of 60° C. The hydrogenation is carried out by pressing on 0.5 MPa hydrogen. Optionally, the temperature rise is limited to 80° C. by way of external cooling. The absorption of hydrogen ends after approximately 4.5 hours.

The catalyst is separated from the reaction batch by way of filtration through a pressure filter that has been preheated to 60° C. to 70° C. and can be prepared for further hydrogenation processes. The reactor and filter are rinsed with approximately 1 liter toluene. The liquid phases are transferred to a reactor, which likewise has been preheated to 60° C. to 70° C.

Crystallization: By applying a vacuum of approximately 100 mbar, the batch is caused to boil and dried azeotropically using an interposed water separator; the return of the toluene is controlled such that ultimately a volume of approximately 6 liters of a dry solution remains in the reactor. The system is then relaxed with nitrogen. The approximately colorless solution that is obtained is cooled to 45° C. using slight external cooling, wherein crystallization takes place. The internal temperature gradually rises by approximately 5° C. during the crystallization process and decreases back to 45° C. after approximately one half to three quarters of an hour. Over a period of 1.5 to 2 hours, cooling down to 5° C. to 10° C. then takes place. The resulting crystallisate is discharged from the reactor and suctioned off, washed with a small amount of cold toluene and then dried at 40° C. to 45° C. under vacuum or under nitrogen in a recirculating dryer. Yield: 1062.6 g (87%) crystals, coarse, colorless to rose-colored, transparent; melting point: 63° C. to 64° C.

### EXAMPLE 2

Hydrogenation: 1108.3 g 7.284 mol) 2-amino-5-nitrotoluene (purity: >99.0%) is added in a nitrogen atmosphere to a solvent mixture consisting of 7290 ml technical xylene and 750 ml ethylbenzene and 660 ml butyl acetate and is stirred for 30 minutes at room temperature. Then, 68 g palladium 10% on carbon (water content 50%, corresponds to 34 g dried product) is added. The batch is then heated to an internal temperature of 60° C. The hydrogenation is carried out by pressing on 0.3 MPa hydrogen. The temperature rise is limited to 85° C. by way of external cooling. The absorption of hydrogen ends after approximately 5 hours.

The catalyst is separated from the reaction batch by way of filtration through a pressure filter that has been preheated to 60° C. to 70° C. and is transferred to a vessel, which previously has likewise been preheated to 60° C. to 70° C. The reactor and filter are rinsed with approximately 1 liter of the solvent mixture.

Crystallization: By applying a vacuum of approximately 75 mbar, the batch is caused to boil and dried azeotropically using an interposed water separator; at the same time, it is concentrated to a volume of approximately 4.5 liters. Then the relaxation with nitrogen is carried out. The approximately colorless solution that is obtained is cooled to 45° C. using slight external cooling, wherein crystallization takes place. The internal temperature gradually rises by approximately 5° C. during the crystallization process and decreases back to 45° C. after approximately thirty minutes. Over a period of 1.5 hours, cooling down to 5° C. to 10° C. then takes place and then the mixture is stirred for another 1 hour at the same temperature. The resulting crystallisate is discharged from the reactor and suctioned off, re-washed with a small amount of cold solvent mixture and then dried at 50° C. under vacuum or under nitrogen in a recirculating dryer. Yield: 758.3 g (85%) crystals, fine, colorless to rose-colored, melting point: 63° C.

The comparison of the analytical data from Examples 1 and 2 supplies identical results and confirms the high purity of the crystallisates.

1. A method for producing 2,5-diaminotoluene, comprising hydrogenating 2-methyl-4-nitroaniline in an aprotic nonpolar solvent or in a mixture made of two or more aprotic nonpolar solvents with hydrogen in the presence of a catalyst or a mixture made of two or more catalysts, thereby forming a solution containing 2,5-diaminotoluene and an aprotic nonpolar solvent or a mixture made of two or more aprotic nonpolar solvents.

2. The method according to claim 1, wherein the aprotic nonpolar solvent or the mixture made of two or more aprotic nonpolar solvents has a dielectric constant ∈ᵣ^{20° C./100 kHz} in the range of 2.3 to 6.1.

3. The method according to claim 1, wherein the catalyst is a solid catalyst or the mixture made of two or more catalysts is a mixture containing at least one solid catalyst.

4. The method according to claim 1, wherein the catalyst is a palladium/activated carbon catalyst or the mixture made of two or more catalysts is a mixture containing at least one palladium/activated carbon catalyst.

5. The method according to claim 1, wherein the 2-methyl-4-nitroaniline is hydrogenated in a temperature range of 0° C. to 150° C.

6. The method according to claim 1, wherein the 2-methyl-4-nitroaniline is hydrogenated at a hydrogen overpressure in the range of 0 to 5 MPa.

7. The method according to claim 1, wherein at least one of the following steps is carried out after the hydrogenation:
separating the catalyst or the mixture made of two or more catalysts from the solution that has been formed;
separating water from the solution that has been formed;
maintaining the temperature of the solution that has been formed in a range in which 2,5-diaminotoluene is precipitated from the solution that has been formed;
separating the precipitated 2,5-diaminotoluene from the solvent or the mixture made of two or more solvents; and
drying the precipitated 2,5-diaminotoluene.

8. The method according to claim 1, further comprising the following steps after the hydrogenation
separating the catalyst or the mixture made of two or more catalysts and water from the solution that has been formed;
maintaining the temperature of the solution that has been formed range in which 2,5-diaminotoluene is precipitated from the solution that has been formed;
separating the precipitated 2,5-diaminotoluene from the solvent or from the mixture made of two or more solvents and
drying the precipitated 2,5-diaminotoluene.

9. The method according to claim 8, wherein precipitation is carried out with crystallization of 2,5-diaminotoluene.

10. Crystalline 2,5-diaminotoluene produced according to claim 9, wherein the 2,5-diaminotoluene is substantially insensitive toward oxidation by air.

11. The crystalline 2,5-diaminotoluene according to claim 9, wherein, using C—K_{α1} radiation, in an x-ray powder diffractogram the 2,5-diaminotoluene shows the strongest reflex in a range of 2Θ=21.5° C. to 22.5° C. and the second-strongest reflex in a range of 2Θ=30.7° C. to 31.7° C.

12. A method of producing cosmetics, dyes, plastic materials or liquid crystals, comprising using the 2,5-diaminotoluene according to claim 10.

13. A method of producing cosmetics, dyes, plastic materials or liquid crystals, comprising using the 2,5-diaminotoluene produced according to claim 8.

14. A method of producing cosmetics, dyes, plastic materials or liquid crystals, comprising using the 2,5-diaminotoluene produced according to claim 9.

15. A method of producing cosmetics, dyes, plastic materials or liquid crystals, comprising using the 2,5-diaminotoluene according to claim 11.
